(19) United States
(12) Patent Application Publication
(10) Pub. No.: US 2023/0310693 A1
(43) Pub. Date: Oct. 5, 2023

(54) Therapeutic And Cosmetic Wound Treatment

(71) Applicant: CYTOSORBENTS CORPORATION, Monmouth Junction, NJ (US)

(72) Inventors: Phillip P. CHAN, Cherry Hill, NJ (US); Ophir ORTIZ, Cherry Hill, NJ (US); Thomas D. Golobish, Princeton, NJ (US); Vincent J. CAPPONI, Monmouth Junction, NJ (US); Wei-Tai Young, Hillsborough, NJ (US)

(21) Appl. No.: 18/022,018

(22) PCT Filed: Aug. 19, 2021
(86) PCT No.: PCT/US2021/046698

§ 371 Date: Feb. 17, 2023

Related U.S. Application Data
(60) Provisional application No. 63/067,417, filed on Aug. 19, 2020.

(51) Int. Cl. A61L 15/44; A61L 15/42; A61L 15/38

(52) U.S. Cl. CPC A61L 15/44; A61L 15/425; A61L 15/38; A61L 2300/254; A61L 2300/43; A61L 2300/402; A61L 2300/414

(57) ABSTRACT

The invention concerns methods for the selective, topical or trans dermal delivery or removal of one or more substances to, or from, respectively, the skin or surface of a human or animal subject, via the topical or transdermal application of a porous, polymeric composition capable of releasing or sorbing (adsorbing and/or absorbing) said substances, wherein said porous polymer is a particulate material, with particles having an average diameter in the range of approximately 0.1 microns to approximately 0.5 centimeters, and having a plurality of pores with an average pore diameter in a range of approximately 50 Angstroms to approximately 40,000 Angstroms.

### CROSS-REFERENCE TO RELATED APPLICATIONS

This application claims priority to and the benefit of U.S. provisional application No. 63/067,417, filed on 19 Aug. 2020, titled “Therapeutic and Cosmetic Wound Treatment,”, the entirety of which is incorporated by reference herein for all purposes.

### TECHNICAL FIELD

The disclosed inventions are in the field of delivering and/or removing one or more substances in a human or animal subject, via topical, extracorporeal, or transdermal application of a porous polymeric material which releases or sorbs (adsorbs and/or absorbs) the substances in question. The disclosed inventions are also in the field of therapeutic, cosmetic, and prophylactic treatments for a human or animal subject.

### BACKGROUND OF THE INVENTION

The skin is a complex, highly active organ that serves many functions. For example, skin acts as an effective protective barrier to infection, moisture, radiation injury, and protection of underlying tissues. Skin helps to regulate body temperature through a variety of means (e.g. perspiration through pores, vasoconstriction or vasodilatation of blood vessels, horripilation, fat insulation, etc.). It is highly innervated, allowing for tactile sensation (e.g. pain, touch, pressure, temperature). The skin is also an immunologically active organ, primed with immune cells and capable of reacting rapidly to injury and infection through the production of cytokines, oxygen radicals, and many other inflammatory mediators. Normal desquamation of cells at the skin surface maintains the healthy appearance and condition of skin.

Skin is composed of 3 general layers. The epidermis is the outermost layer of skin composed of cells (e.g. keratinocytes) that provide a waterproof infection barrier (via stratum corneum and surface oils), protection from the sun via pigmented cells (e.g. melanocytes, melanin), and allergen and infection surveillance (e.g. Langerhans cells, CD8 T-cells, etc.). Langerhans cells are modified dendritic cells that have projections that reach the skin surface, allowing them to sample antigen and sense skin flora and toxins, resulting in either an up-regulation or down-regulation of inflammation in the epidermis and dermis. Contact dermatitis due to urushiol toxin (e.g. poison ivy), is one such example. The ability to “sample the environment” is important to the overall health of the skin. It occurs at the skin surface, or stratum corneum, where changing the chemical or biologic composition of the skin surface may be a novel strategy to alter inflammation in the dermis and epidermis. Normal desquamation of the stratum corneum is also important in the appearance and health of skin. Terminally differentiated keratinocytes, or corneocytes, make up the bulk of the stratum corneum, where mechanical shear and proteolytic degradation of intercellular connections at desmosomes between cells, leads to sloughing off of corneocytes and normal desquamation. A failure of this process can lead to accumulation of corneocytes, or hypercornification, which can contribute to pore clogging and acne, dry skin, dandruff, and other skin problems, for example. Psoriasis, is another disorder of the epidermis, caused by the premature maturation and overproliferation of keratinocytes induced by an inflammatory cascade in the dermis driven by pro-inflammatory cytokines.

The dermis is the next layer that is composed of cells (e.g., fibroblasts, CD4 lymphocytes, dendritic cells and other immune cells), blood vessels, nerve endings, sweat glands that secrete water, electrolytes and other chemicals, hair follicles with sebaceous glands that secrete sebum and other oils (pilosebaceous unit), and structural proteins such as collagen, elastin and fibrillin. The structural architecture of the dermis gives the skin its flexibility and strength. Acne is a common dermal condition caused by acute and chronic inflammation of the pilosebaceous unit caused by increased sebum production, bacterial overgrowth, abnormal desquamation, and other factors that can lead to pain, discomfort and scarring.

The third layer of skin is the subcutaneous fat layer that provides insulation and shock absorption.

As the body's first line defense, the skin is exposed to many sources of potential injury, irritation, or infection. In many cases, the source of tissue injury is external (e.g. heat, cold, external trauma such as crush injury or laceration, toxin-mediated, electromagnetic radiation, electrical injury) or intrinsic to the body (e.g. mediated by cells, antibodies, enzymes, cytokines, complement factor, oxygen radicals, histamines and other inflammatory mediators, etc.). The source of irritation could be noxious agents (e.g. shear stress, oils, sebum, comedones, fatty acids, peptides, proteins, prostaglandins, derivatives of arachidonic acid, oxygen radicals, cosmetics, etc.). The source of infection could be bacterial, viral, fungal, parasitic, prion-related, or other, for example.

An understanding of skin architecture and how it responds to injury, infection, irritation, and inflammation and age is important because it highlights the potential points of intervention of a topical therapy in many different applications such as wound healing, pain reduction, cosmetics (e.g. skin rejuvenation and anti-aging), and treatment of dermatologic conditions (e.g. acne). The following representative examples provide the rationale to use of a topical or transdermal biocompatible porous polymer therapy that can either deliver or remove substances from the skin or body surfaces. The use of such therapies extends well beyond these specific examples.

Wound healing: When skin and the underlying tissue is injured and creates an open wound, healing occurs through an orderly, but complex series of steps, involving the interaction of many families of cells (e.g. fibroblasts, macrophages, etc.), molecules (e.g. growth factors, cytokines, inflammatory modulators, matrix metalloproteinases and other enzymes), and other factors. See, e.g., Enoch, et al., “ABCs of Wound Healing Recent Advances and Emerging Treatments,” BMJ, 332:962-965 (2006); Olmarker, et al., “Inhibition of Tumor Necrosis Factor May Improve Wound Healing and Reduce Scar Formation Following Laminectomy. A Pilot Study In Pigs,” Internet J. Spine Surgery JSSN: 1937-8270; Schietz, et al., “Molecular Analysis of the Environments of Healing and Chronic Wounds: Cytokines, Proteases and Growth Factors,” Primary Intervention, 7-14 (February 1999); Cytokines in Wound Healing. (R&D Systems Catalog, 2002); Forsberg, et al., “Correlation of Procalcitonin and Cytokine Expression with Dehiscing of wartime extremity wounds,” J. Bone Joint Surg. Am., 90(3):580-588 (2008); Finnerty, et al., “Temporal Cytokine Profiles in Severely Burned Patients: A Comparison of Adults and Children,” Mol. Med., 14(9-10):553-5560 (2008), all of which are incorporated by reference in their entirety.

What the literature teaches the skilled artisan is that wound healing is a process involving timely progression through three stages: inflammation, new tissue formation, and remodeling, accompanied by the presence of both beneficial and harmful substances for wound repair. The transition from one stage to another is dependent upon maturation and differentiation of different cell population such as keratinocytes, fibroblasts and macrophages that are involved in the repair process. The first step in the inflammation phase is the formation of a blood clot by activated platelets. Large amounts of cytokines are either produced, released, or induced by activated platelets, endothelial cells and other damage-associated molecular patterns (DAMPs) and pathogen-associated molecular patterns (PAMPs), for example. Subsequently, cytokines attract neutrophils that then undergo apoptosis and release additional cytokines. Most chronic wounds exhibit a defect in the progression from the inflammation stage, to the new tissue formation stage where cell maturation and differentiation ensue (Loots M A, Lamme E N, Zeegelaar J, Mekkes J R, Bos J D and Middelkoop E. Differences in cellular infiltrate and extracellular matrix of chronic diabetic and venous ulcers versus acute wounds. J Invest Dermatol 1998; 111: 850-857). This is also true in diabetic mice where the chronic wound is characterized by prolonged expression of cytokines and massive infiltration of wound associated macrophages (Wetzler C, Kampfer H, Stallmeyer B, Pfeilschifter J and Frank S. Large and sustained induction of chemokines during impaired wound healing in the genetically diabetic mouse: prolonged persistence of neutrophils and macrophages during the late phase of repair. J Invest Dermatol 2000; 115: 245-253.) A typical healing wound often demonstrates increased mitogenic activity of cells with a reduction of inflammatory cytokines and proteases in and around the wound over time. A typical chronic ulcer often has low mitogenic activity and senescent cells, with high concentrations of inflammatory cytokines and proteases in and around the wound.

Approaches to improve acute and chronic wound healing include for example, the use of antibiotics, wet to dry gauze dressing changes, physical debridement, vacuum dressings, use of artificial or bioengineered skins, revascularization of the affected area, growth factors and other biologics, plasma concentrates, gene-based or cell-based therapies. Reduction of inflammatory mediators and other harmful substances during the inflammation phase with a novel porous polymer sorbent would represent a new approach to augment wound healing by priming a timely transition to the tissue formation and wound remodeling stages.

Scar modification: One of the results of wound healing is the formation of scar tissue in the injured area. Scar tissue is typically fibrous connective tissue that replaces the normal tissue architecture. Scar tissue can be structurally inferior to the original tissue, painful, and cosmetically unappealing, and may not provide adequate wound healing. Hypertrophic scarring and keloid formation are examples of excessive scar formation. The formation of scar tissue is the combined result of local inflammation driven by cytokines and chemokines, the types of cells recruited and present in the wound, abnormal collagen production and deposition (e.g. Collagen type-I versus Collagen type-III), with cross-linking by enzymes such as lysyl hydroxylase-2b, as well as the production and secretion of other molecules by recruited cells such as osteopontin by fibroblasts, and TGF-β1 and TGF-β2 by wound associated macrophages, platelets and epidermal cells, for example. This complex network of activity offers multiple strategies to potentially reduce scar formation. For example, balanced modulation of cytokines and chemokines is associated with scarless wound healing [Liechty K W, Adzick N S, Crombleholme T M. [Diminished interleukin 6 (IL-6) production during scarless human fetal wound repair. Cytokine, 2000. 12(6): 691-8]. The topical reduction, but not complete elimination, of substances such as collagen, collagen-crosslinking enzymes such as lysyl hydroxylase-2b, and cytokines or other inflammatory mediators during the wound healing process or afterwards, represents a novel strategy in reducing normal or excessive scar formation such as keloids and hypertrophic scars.

Treatment of Acne: Inflammation of the skin can also be seen in the absence of physical trauma. Acne vulgaris, for example, is one such well-known example. When skin pores become occluded (e.g. with oil, sebum, or epithelial cells), contaminated with bacteria (e.g. Propionibacterium acnes, Staphylococcus aureus, etc.), or irritated (e.g. due to saturated or unsaturated fatty acids, oils, or physical trauma) inflammation can result as well. Traumatic decompression of acne lesions and healing of acne lesions can cause atrophic or hypertrophic scarring and pigmented discoloration such as post-inflammatory erythema (PIE) or post-inflammatory hyperpigmentation (PIH). In such conditions, the use of a topical porous polymeric sorbent material to adsorb causative inflammatory substances, such as oils, fatty acids, or bacterial toxins, may prevent or treat acne, ameliorate the inflammation and pain, reduce scarring, reduce PIE or PIH, improve tissue remodeling, and promote healing. When this polymeric sorbent is in a particulate form (e.g. beads), the material has the added benefit of being an exfoliant or a microdermabrasive material that can help clear pores. When the polymeric sorbent is mixed with or pre-loaded with medications or chemicals such as cleansers, moisturizers, sunscreens, antibiotics, benzoyl peroxide, alpha-hydroxy acids (such as glycolic acid, lactic acid, citric acid and mandelic acid), beta-hydroxy acids such as salicylic acid, hydroxyquinone, or others, it can work in conjunction with the medications, chemicals, or drugs, including delivering them to the skin and treating acne lesions.

Rosacea: Rosacea is a chronic condition characterized by erythema and chronic inflammation primarily in the face and neck that can lead to hyperplasia, nodular or pustular swelling, erythema, and capillary congestion of the skin of the face and nose. While there is no cure for rosacea and the etiology of the disease is unknown, symptomatic treatment with oral and topical antibiotics and anti-inflammatory treatments have been useful. The use of a topical porous polymer sorbent to remove causative inflammatory mediators, putative bacterial toxins, and irritating chemicals and oils from the affected skin may result in symptomatic or definitive treatment.

Skin rejuvenation, anti-aging and anti-wrinkling: Visible skin aging, including wrinkles and fine lines, are associated with chronic cutaneous inflammation. Kligman and Lavker documented that inflammation causes microscopic effects in visible skin aging [R Lavker and A Kligman, Chronic Heliodermatitis: A morphological evaluation of chronic actinic dermal damage with emphasis on the role of mast cells. J Invest Derm 90, 325-330 (1988)]. This microscopic chronic inflammation can be triggered in many ways, such as mild or repeated skin barrier disruption, sub-erythematous exposure to UV light, skin flora, chemical exposure, and other factors. [P Elias et al, The link between barrier function and inflammation. Arch Derma 137(8), 60-62 (2001)] & [L Baumann, L Eichenfield and S Taylor, Advancing the Science of Naturals. Cosm Dermatol 18(24) suppl 4, 2-7 (2005)]. For example, skin barrier disruption activates the release of TNF-α, IL-1, IL-8, and other pro-inflammatory cytokines [B Nickoloff and Y Naidu, Perturbation of epidermal barrier function correlates with initiation of cytokine cascade in human skin. J Am Acad Derm 30 535-546 (1994)] & [P Elias et al, Barrier function regulates DNA synthesis. Sem Dermatol 11 176-182 (1992)]. Inflammation leads to the production and activation of matrix metalloproteinases (MMPs) by fibroblasts, keratinocytes and mast cells, normally important in remodeling of the skin post-injury, but in chronic inflammation can exacerbate inflammation and degrade dermal components such as collagen and elastin, leading to wrinkles and aging in skin. Important MMPs include Collagenase (MMP-1), stromelysin (MMP-3) and gelatinase (MMP-9). Visible skin aging features, such as fine lines, wrinkles, fragility and laxity are due to solar elastosis, collagen destruction and tissue atrophy induced by the damaging activities of these MMPs [B Pilcher et al, Collagenase-1 and collagen in epidermal repair. Arch Derma Res 290 suppl 37-46 (1998)]. The topical application of a biocompatible, porous polymer sorbent has the potential ability to remove surface inflammatory mediators of chronic inflammation and cutaneous MMPs, thereby reducing the activity of MMPs and other cutaneous inflammatory mediators as well as potentially impact Langerhans cells and other cells involved in sampling and inflammation of the skin. When used over a period of time, the porous polymer sorbent may help to prevent or treat chronic cutaneous inflammation to reverse or prevent visible skin aging, including wrinkle formation, fine lines and skin rejuvenation over time. These porous polymers are distinct from commercially available exfoliating solid beads found in common cosmetic products as they have demonstrated the ability to actively bind and remove inflammatory mediators such as cytokines and bioactive lipids.

Skin cancer: Chronic cutaneous inflammation due to skin barrier disruption is linked to skin cancer as well. Halliday reported a strong correlation between prolonged use of topical retinoids (that causes skin disruption) with an increased incidence of skin cancer [GM Halliday et al, J Invest Derm 114(5) 923-7 (2000)]. Similarly, inflammatory MMP enzymes play important roles in the pre-malignant and malignant damage to skin cells [B Pilcher et al, Collagenase-1 and collagen in epidermal repair. Arch Derma Res 290 suppl 37-46 (1998)]. Proper desquamation and cell turnover is also important. A topical porous polymer sorbent may help in normal skin turnover and removal of inflammatory mediators from the skin, may help prevent the risk of skin cancer by reducing chronic inflammation that is associated with an increased risk of malignancy.

The invention which follows is based upon the discovery that porous polymeric particles may be formulated into compositions that either deliver or remove substances to or from the skin and underlying tissues, in order to modulate skin inflammation. Such compositions, for example, can contain porous polymer sorbent particles that can remove inflammatory substances and excipients that aid in controlling inflammation and infection, or promote healing. In doing so, these porous, polymeric compositions may be capable of improving wound healing, preventing skin inflammation, damage and aging, improving cosmesis, and treating a wide range of dermatologic conditions.

### SUMMARY OF THE INVENTION

In some embodiments, the invention concerns methods for the selective, topical or transdermal delivery or removal of one or more substances to, or from, respectively, the skin or surface of a human or animal subject, via the topical or transdermal application of a porous, polymeric composition capable of releasing or sorbing (adsorbing and/or absorbing) said substances, wherein said porous polymer is a particulate material, with particles having an average diameter in the range of approximately 0.1 microns to approximately 0.5 centimeters, and having a plurality of pores with an average pore diameter in a range of approximately 50 Angstroms to approximately 40,000 Angstroms.

Some embodiments concern the application of said porous polymeric composition to a wound. In certain embodiments, the wound is a chronic wound, a venous stasis ulcer, an ulcer or ulceration caused by a disease, a traumatic wound, a burn wound, a diabetic wound, or a surgical wound.

The invention may concern the application of said porous polymeric composition to alter or improve wound healing, reduce scarring, improve tissue remodeling, or reduce inflammation or pain. Treatment may comprise the application of said porous polymeric composition to skin. Treatment may comprise the application of said porous polymeric composition to skin graft site.

Some treatments comprise the transdermal application of said porous polymeric composition for plastic, cosmetic, and reconstructive surgery applications. Other treatments comprise skin application is for cosmetic applications. Cosmetic applications may include one or more of skin brightening, cleansing, exfoliating, anti-aging, beautifying, anti-wrinkle, softening, oil reduction, pore cleansing, skin rejuvenation, improving skin discoloration, and reducing fine lines, skin laxity, and skin fragility.

Skin application may also comprise treatment of one or more dermatologic conditions. Dermatological conditions may include infections, yeast infection, fungal infection, warts, malodorous skin, hyperhidrosis, dandruff, seborrheic dermatitis, skin manifestations of autoimmune diseases, psoriasis, lupus, Lichen planus, dry or oily skin, eczema, atopic dermatitis, contact or allergic dermatitis, tinea, vitiligo, rashes, hives, decubitus ulcers, canker or cold sores, stomatitis, versicolor, pemphigoid, rosacea, skin blotchiness, corns, calluses, ichthyosis vulgaris, keloids, seborrheic keratosis, actinic keratosis, and skin cancer. In one embodiment the dermatological condition is acne vulgaris.

In certain embodiments, skin application alters the activity of immune cells, such as Langerhan's cells, in the skin. The change in activity of immune cells, such as Langerhan's cells, may lead to a change in inflammation or aging of the skin.

In some embodiments, the porous polymeric composition is in the form of a powder, poultice, mask, a liquid, gel paste, a low volume paste, a gel, a dispersion, a slurry, or a suspension. The porous polymeric composition may be used in conjunction with a cleansing cloth, pad, towelette, or rotary cleansing apparatus.

In some embodiments, the porous polymeric composition also comprises a permeable material or liquid. Permeable material comprise gauze, mesh, pad, or permeable or semi-permeable membrane in which said porous polymeric material is embedded, or enclosed (e.g. as in a pouch, or bandage).

In certain embodiments, the permeable material is a liquid, gel, lotion, or paste. The permeable material may comprise one or more chemicals (e.g. drugs, medications, vitamins, nitric oxide, nitric oxide donors), minerals, or nutrients. Suitable chemicals include an antibiotic, anti-viral, antifungal, or anti-parasitic medicine.

In some embodiments, the permeable material contains at least one cosmetic ingredient. Cosmetic ingredient include one or more of glycerin, hyaluronic acid, a hyaluronic acid salt, shea butter, vitamins, vitamin E, vitamin A, vitamin D, vitamin C, and vitamin K. Cosmetic ingredients may also comprise one or more of cleansers, moisturizers, sunscreens, antibiotics, benzoyl peroxide, keratolytic agents, alpha-hydroxy acids (such as glycolic acid, lactic acid, citric acid and mandelic acid), beta-hydroxy acids (such as salicylic acid), retinoic acid, hydroxyquinone, potassium hydroxide, tea extracts, and plant and flower extracts.

In certain embodiments, substance to be removed is a protein, peptide, glycosylated protein (e.g. advanced glycation end product), or protein containing molecule. The substance may comprise a protein-based inflammatory mediator such as a cytokine (e.g. TNF-α, IL-1, IL-4, IL-5, IL-6, IL-8, IL-10, IL-12, RANTES, MCP-1, or IP-10.), toxin, or activated complement. In other embodiments, the substance is a growth factor (e.g. epidermal growth factor, fibroblast growth factor, platelet derived growth factor, transforming growth factor, vascular endothelial growth factor). In yet other embodiments, the substance is an enzyme. Enzymes include one or more of a metalloproteinase, a collagenase, an elastase, or a cross-linking enzyme. Substances to be removed may also comprise one or more of a non-protein inflammatory mediator such as a prostaglandin, leukotriene, bioactive lipid (e.g. eicosanoid, endocannabinoid, and sphingolipid) or histamine. Other substances comprise one or more of a hormone, a pain mediator or a wax, squalene, fatty acid, triglyceride, or oil, such as sebum, or urushiol oil.

In some embodiments, the porous polymeric composition comprises a mixture of particles having at least two different average diameters.

Preferably, the porous polymeric material is biocompatible or does not induce inflammation.

In some preferred embodiments, the porous polymeric material comprises a plurality of pores ranging from 50 Å to 40,000 Å with a pore volume of 0.5 cc/g to 5.0 cc/g, said sorbent having a size of 0.05 mm to 2 cm;

wherein the sorbent has a pore structure such that the total pore volume of pore size in the range of 50 Å to 40,000 Å is greater than 0.5 cc/g to 5.0 cc/g dry sorbent; wherein:
(i) the ratio of the total pore volume of pore diameter in the range of 50 Å to 40,000 Å to the total pore volume of pore diameter in the range of 100 Å to 1,000 Å of the sorbent is smaller than 3:1; or
(ii) the ratio of the total pore volume of pore diameter in the range of 50 Å to 40,000 Å to the total pore volume of pore diameter in the range of 1,000 Å to 10,000 Å of the sorbent is smaller than 2:1; or
(iii) the ratio of the total pore volume of pore diameter in the range of 50 Å to 40,000 Å to the total pore volume of pore diameter in the range of 10,000 Å to 40,000 Å of the sorbent is smaller than 3:1.

### BRIEF DESCRIPTION OF THE DRAWINGS

FIG. 1 depicts a 6 well plate with select wells (gray) filled with Cytochrome C solution for the study described in Example 1.

FIG. 2 depicts a plot of % Cytochrome C Remaining vs. time for coarse mesh packs (lower curve) and fine mesh packs (upper curve) for the study described in Example 1.

FIG. 3 depicts 8 mm diameter full thickness skin wounds that were made on the backs of dead rats for the study described in Example 2.

FIG. 4 depicts a qualitative comparison of skin wound healing rates after 14 days for 8 mm diameter full thickness skin wounds created surgically under anesthesia on the backs of rats. The top figure represents porous test beads. The middle figure represents non-porous beads. The lower figure represents non-treated wounds.

FIG. 5 depicts a qualitative comparison of adsorption of hemoglobin protein 4 hours after surgery to create the wounds. The top figures (A) show the results of using test beads and the bottom figures (B) show the results of using non-porous control beads.

### DETAILED DESCRIPTION OF ILLUSTRATIVE EMBODIMENTS

The porous polymeric compositions of the invention comprise biocompatible particles which have an average diameter of from about 0.1 microns to about 2 centimeters, more preferably from about 0.1 microns to about 1 centimeters, and most preferably from about 0.1 microns to about 0.75 centimeters. Those of ordinary skill in the art will recognize that particulate compositions at the smaller end of these ranges will appear as powders, while those at the larger end will appear as beads. In practice, the compositions of the invention may consist of particles which are essentially uniform in diameter or may be a mixture of particles of different diameters, with the proportions of each size particle being determined by the application for which the particles are intended.

As noted, supra, the particles are porous. The pores of the particles may range in average diameter from about 50 Angstroms to about 40,000 Angstroms, more preferably from 50 to 20,000 Angstroms, to most preferably from 50 to 5,000 Angstroms. The total volume of the pores ranges from about 0.5 cc to about 3.0 cc per gram of dry polymer. “Dry” polymer as used herein is elaborated upon infra.

The polymers used to make the compositions of the invention may be biocompatible, and when used, e.g., in the treatment of wounds or other conditions where they contact blood, hemocompatible. U.S. Pat. Nos. 7,875,182 and 7,846,650, both of which are incorporated by reference in their entirety, provide examples of biocompatible and hemocompatible polymers useful in the subject invention. The skilled artisan will be aware of others which need not be set forth herein.

In practice, the particles of the invention, in view of their priority, permit selective adsorption of molecules involved in inflammation so as to accelerate wound healing, or other biological phenomena where removal of harmful molecules can expedite the healing process. Exemplary of the molecules which can be removed with the compositions of the invention are cytokines, inflammatory modulators, enzymes, hormones, pain mediators, and other substances which the skilled artisan will recognize as being harmful to the healing and/or recovery processes. Specific examples of these categories of molecules include TNF, IL-1, IL-4, IL-5, IL-6, IL-8, IL-10, IL-12, RANTES, MCP-1, and IP-10 for cytokines, metalloproteinases, collagenases, elastases, collagen crosslinking agents for enzymes, substance P, serotonin, histamine, C-GRP, bradykinin, prostaglandins, and arachidonic acids, cytochrome C, as well as interleukins and pain mediators. Where more than one form of a particular molecule is known, as is frequently the case for, e.g., interleukins and other cytokines, all forms of the molecule are encompassed herein.

Optionally, the particles of the inventive composition may be coated with additional materials to help to facilitate the sorption (adsorption or absorption) of the targeted, harmful substances. An exemplary, but by no means comprehensive list of materials which can be placed on the particles and/or in the pores there are sulfonated polymers and hydroxylated polymers, lactose, N-acetylglucosamine, antibiotics (e.g., beta lactams, bacitracin, etc.), antibodies, as well as ligands for harmful substances.

Active forms of these molecules, such as binding fragments of antibodies and ligands, proteins of complex glycoproteins or sugars, and so forth, are contemplated as part of the invention as well.

The particles are constructed, following methods well known in the art, and can be said to be “semi-selective.” By “semi-selective” essentially two concepts are meant. First, the adsorptive properties are such that larger, beneficial molecules, such as antibodies (which have an average molecular weight of 150 kilodaltons or more), will not be adsorbed or are minimally adsorbed. Second, the particles will adsorb most molecules which fall within a specific range, e.g., 10-85 kilodaltons while selectivity can be improved, e.g., by selecting an appropriate coating as discussed supra, the particles will not remove all of any potentially beneficial molecule, and will remove some, but not all beneficial molecules which fall within the operative range of the particles. In a preferred embodiment, the particles are made such that they sorb molecules having a molecular weight of from about 10 to about 85 kilodaltons. The skilled artisan will note from the above description, that particles capable of sorbing smaller and/or larger molecules may be designed, and mixtures of particles having varied sorptive properties are contemplated. As a result of these properties, when the particles are used therapeutically, e.g., in treating wounds, they can be applied at points in time, such as the inflammatory phase, when problematic molecules are prevalent, and then removed.

In addition to the treatment of wounds, the invention can be used for conditions where treatment can be seen as straddling therapeutic and cosmetic applications. Exemplary of these, but by no means the only such conditions, are infections, ulcerations caused by Herpes simplex virus, warts, malodorous skin, hyperhidrosis, dandruff, seborrheic dermatitis, skin manifestations of autoimmune diseases, psoriasis, lupus, Lichen planus, dry or oily skin, any and all forms of eczema, atopic dermatitis, contact or allergic dermatitis, tinea, vitiligo, rashes, hives, decubitus ulcers, canker or cold sores, stomatitis, versicolor, pemphigoid, rosacea, corns, calluses, ichthyosis vulgaris, keloids, seborrheic keratosis, actinic keratosis, and skin cancer. Other examples include skin blotchiness, pore rejuvenation and pore cleansing, prevention or reduction of wrinkles, hair regrowth, skin discoloration, skin rejuvenation and anti-aging.

Delivery of beneficial substances to the skin or wound is also possible with this invention. Porous polymers can also be loaded with drugs or medications (e.g. antibiotics, alpha-hydroxy acids (such as glycolic acid, lactic acid, citric acid and mandelic acid), beta-hydroxy acids (such as salicylic acid), hydroxyurea, retinoic acid, salicylic acids, benzoyl peroxide, hydroxyquinone, nitric oxide donors, keratolytic agents, and anti-viral medications), as well as vitamins, minerals and other nutrients. The porous polymers can also be formulated into compositions containing similar substances, and then applied to the skin or wound.

The compositions of the invention may also be used in purely cosmetic applications. In such situations, the particles are combined with at least one cosmetic product such as a cleanser, mask, emollient or moisturizer. Exemplary additives include glycerin, hyaluronic acid, or a salt thereof, shea butter, vitamins, benzoyl peroxide, keratolytic agents, alpha-hydroxy acids (such as glycolic acid, lactic acid, citric acid and mandelic acid), beta-hydroxy acids (such as salicylic acid), retinoic acid, hydroxyquinone, potassium hydroxide, tea extracts, and plant and flower extracts.

The composition of the invention may be modified on the polymer surface to incorporate metal ion or other surface coating (e.g. zinc, selenium disulfide, and ketoconazole, silver) that can promote antimicrobial activity. Alternatively, polymers may serve as a vehicle for metal ion or other compound delivery (e.g. zinc pyrithione delivery). In such cases, the utility of the invention would be to treat acne vulgaris, abscesses, yeast infection, dandruff, and seborrheic dermatitis. It can also have antibacterial properties and is effective against many pathogens from the Streptococcus and Staphylococcus genera. Other medical applications may be treatments of psoriasis, eczema, athletes foot, dry skin, atopic dermatitis, tinea, and vitiligo in topical applications.

Some preferred polymers comprise residues from one or more monomers, or containing monomers, or mixtures thereof, selected from acrylonitrile, allyl glycidyl ether, butyl acrylate, butyl methacrylate, cetyl acrylate, cetyl methacrylate, 3,4-dihydroxy-1-butene, dipentaerythritol diacrylate, dipentaerythritol dimethacrylate, dipentaerythritol tetraacrylate, dipentaerythritol tetramethacrylate, dipentaerythritol triacrylate, dipentaerythritol trimethacrylate, divinylbenzene, divinylformamide, divinylnaphthalene, divinylsulfone, 3,4-epoxy-1-butene, 1,2-epoxy-9-decene, 1,2-epoxy-5-hexene, ethyl acrylate, ethyl methacrylate, ethylstyrene, ethylvinylbezene, glycidyl methacrylate, methyl acrylate, methyl methacrylate, octyl acrylate, octyl methacrylate, pentaerythritol diacrylate, pentaerythritol dimethacrylate, pentaerythritol tetraacrylate, pentaerythritol tetramethacrylate, pentaerythritol triacrylate, pentaerythritol trimethacrylate, styrene, trimethylolpropane diacrylate, trimethylolpropane dimethacrylate, trimethylolpropane triacrylate, trimethylolpropane trimethacrylate, trivinylbenzene, trivinylcyclohexane, vinyl acetate, vinylbenzyl alcohol, 4-vinyl-1-cyclohexene 1,2-epoxide, vinylformamide, vinylnaphthalene, 2-vinyloxirane, and vinyltoluene.

Some embodiments of the invention use an organic solvent and/or polymeric porogen as the porogen or pore-former, and the resulting phase separation induced during polymerization yield porous polymers. Some preferred porogens are selected from, or mixtures comprised of any combination of, benzyl alcohol, cyclohexane, cyclohexanol, cyclohexanone, decane, dibutyl phthalate, di-2-ethylhexyl phthalate, di-2-ethylhexylphosphoric acid, ethylacetate, 2-ethyl-1-hexanoic acid, 2-ethyl-1-hexanol, n-heptane, n-hexane, isoamyl acetate, isoamyl alcohol, n-octane, pentanol, poly(propylene glycol), polystyrene, poly(styrene-co-methyl methacrylate), tetraline, toluene, tri-n-butylphosphate, 1,2,3-trichloropropane, 2,2,4-trimethylpentane, and xylene.

In yet another embodiment, the dispersing agent is selected from a group consisting of hydroxyethyl cellulose, hydroxypropyl cellulose, poly(diethylaminoethyl acrylate), poly(diethylaminoethyl methacrylate), poly(dimethylaminoethyl acrylate), poly(dimethylaminoethyl methacrylate), poly(hydroxyethyl acrylate), poly(hydroxyethyl methacrylate), poly(hydroxypropyl acrylate), poly(hydroxypropyl methacrylate), poly(vinyl alcohol), salts of poly(acrylic acid), salts of poly(methacrylic acid) and mixtures thereof.

Preferred sorbents are biocompatible. In another further embodiment, the polymer is biocompatible. In yet another embodiment, the polymer is hemocompatible. In still a further embodiment the biocompatible polymer is hemocompatible. In still a further embodiment the geometry of the polymer is a spherical bead.

In another embodiment, the biocompatible polymer comprises poly(N-vinylpyrrolidone).

In another embodiment, the biocompatible polymer comprises 1,2-diols. In another embodiment, the biocompatible polymer comprises 1,3-diols

In another further embodiment, the biocompatible polymer comprises heparin mimicking polymers.

The coating/dispersant on polymer material will imbue the material with improved biocompatibility.

Some preferred polymers comprise residues of one or more of divinylbenzene, ethylvinylbezene, styrene, and ethylstyrene monomers. One preferred polymer is the poly(styrene-co-divinylbenzene) resin.

In still yet another embodiment, a group of cross-linkers consisting of dipentaerythritol diacrylates, dipentaerythritol dimethacrylates, dipentaerythritol tetraacrylates, dipentaerythritol tetramethacrylates, dipentaerythritol triacrylates, dipentaerythritol trimethacrylates, divinylbenzene, divinylformamide, divinylnaphthalene, divinylsulfone, pentaerythritol diacrylates, pentaerythritol dimethacrylates, pentaerythritol tetraacrylates, pentaerythritol tetramethacrylates, pentaerythritol triacrylates, pentaerythritol trimethacrylates, trimethylolpropane diacrylate, trimethylolpropane dimethacrylate, trimethylolpropane triacrylate, trimethylolpropane trimethacrylate, trivinylbenzene, trivinylcyclohexane and mixtures thereof can be used in formation of a hemocompatible hydrogel coating.

In some embodiments, the polymer is a polymer comprising at least one crosslinking agent and at least one dispersing agent. The dispersing agent may be biocompatible. The dispersing agents can be selected from chemicals, compounds or materials such as hydroxyethyl cellulose, hydroxypropyl cellulose, poly(diethylaminoethyl acrylate), poly(diethylaminoethyl methacrylate), poly(dimethylaminoethyl acrylate), poly(dimethylaminoethyl methacrylate), poly(hydroxyethyl acrylate), poly(hydroxyethyl methacrylate), poly(hydroxypropyl acrylate), poly(hydroxypropyl methacrylate), poly(vinyl alcohol), salts of poly(acrylic acid), salts of poly(methacrylic acid) and mixtures thereof; the crosslinking agent selected from a group consisting of dipentaerythritol diacrylates, dipentaerythritol dimethacrylates, dipentaerythritol tetraacrylates, dipentaerythritol tetramethacrylates, dipentaerythritol triacrylates, dipentaerythritol trimethacrylates, divinylbenzene, divinylformamide, divinylnaphthalene, divinylsulfone, pentaerythritol diacrylates, pentaerythritol dimethacrylates, pentaerythritol tetraacrylates, pentaerythritol tetramethacrylates, pentaerythritol triacrylates, pentaerythritol trimethacrylates, trimethylolpropane diacrylate, trimethylolpropane dimethacrylate, trimethylolpropane triacrylate, trimethylolpropane trimethacrylate, trivinylbenzene, trivinylcyclohexane and mixtures thereof. Preferably, the polymer is developed simultaneously with the formation of the coating, wherein the dispersing agent is chemically bound or entangled on the surface of the polymer.

In still another embodiment, the biocompatible polymer coating is selected from a group consisting of poly(diethylaminoethyl methacrylate), poly(dimethylaminoethyl methacrylate), poly(hydroxyethyl acrylate), poly(hydroxyethyl methacrylate), poly(hydroxypropyl acrylate), poly(hydroxypropyl methacrylate), poly(N-vinylpyrrolidone), poly(vinyl alcohol), salts of poly(acrylic acid), salts of poly(methacrylic acid) and mixtures thereof.

In still another embodiment, the biocompatible oligomer coating is selected from a group consisting of poly(diethylaminoethyl methacrylate), poly(dimethylaminoethyl methacrylate), poly(hydroxyethyl acrylate), poly(hydroxyethyl methacrylate), poly(hydroxypropyl acrylate), poly(hydroxypropyl methacrylate), poly(N-vinylpyrrolidone), poly(vinyl alcohol), salts of poly(acrylic acid), salts of poly(methacrylic acid) and mixtures thereof.

Some present biocompatible sorbent compositions are comprised of a plurality of pores. The biocompatible sorbents are designed to adsorb a broad range of toxins from less than 0.5 kDa to 1,000 kDa. While not intending to be bound by theory, it is believed the sorbent acts by sequestering molecules of a predetermined molecular weight within the pores. The size of a molecule that can be sorbed by the polymer will increase as the pore size of the polymer increases. Conversely, as the pore size is increased beyond the optimum pore size for adsorption of a given molecule, adsorption of said protein may or will decrease.

In certain methods, the solid form is porous. Some solid forms are characterized as having a pore structure having a total volume of pore sizes in the range of from 10 Å to 40,000 Å greater than 0.1 cc/g and less than 5.0 cc/g dry polymer.

The pore structures of the adsorbent polymers were analyzed with either a Micromeritics ASAP 2020 instrument (N2 adsorption/desorption isotherms) or a Micromeritics AutoPore IV 9500 (Mercury Intrusion Porosimeter). For N2 sorption data, the total pore volume and size distribution were calculated based on the desorption branch of isotherms using the Barrett-Joyner-Halenda (BJH) method. From the pressure versus intrusion data, the Mercury intrusion Porosimeter generates volume and size distributions using the Washburn equation. In some preferred embodiments, the sorbent is porous and the ratio of pore volume between 50 Å to 40,000 Å (pore diameter) to pore volume between 1,000 Å to 10,000 Å (pore diameter) of the sorbent is smaller than 3:1, 6:1, 8:1 or 10:1.

In certain embodiments, the polymers can be made in bead form having a diameter in the range of 0.1 micrometers to 2 centimeters. Certain polymers are in the form of powder, beads or other regular or irregularly shaped particulates.

In some embodiments, the plurality of solid forms comprises particles having a diameter in the range for 0.1 micrometers to 2 centimeters.

In some embodiments, sorbents include cross-linked polymeric material derived from the reaction of a cross-linker with one or more of the following polymerizable monomers, then subsequently epoxidized and ring-opened to form a polyol: acrylonitrile, allyl glycidyl ether, butyl acrylate, butyl methacrylate, cetyl acrylate, cetyl methacrylate, 3,4-dihydroxy-1-butene, dipentaerythritol diacrylate, dipentaerythritol dimethacrylate, dipentaerythritol tetraacrylate, dipentaerythritol tetramethacrylate, dipentaerythritol triacrylate, dipentaerythritol trimethacrylate, divinylbenzene, divinylformamide, divinylnaphthalene, divinylsulfone, 3,4-epoxy-1-butene, 1,2-epoxy-9-decene, 1,2-epoxy-5-hexene, ethyl acrylate, ethyl methacrylate, ethylstyrene, ethylvinylbezene, glycidyl methacrylate, methyl acrylate, methyl methacrylate, octyl acrylate, octyl methacrylate, pentaerythritol diacrylate, pentaerythritol dimethacrylate, pentaerythritol tetraacrylate, pentaerythritol tetramethacrylate, pentaerythritol triacrylate, pentaerythritol trimethacrylate, styrene, trimethylolpropane diacrylate, trimethylolpropane dimethacrylate, trimethylolpropane triacrylate, trimethylolpropane trimethacrylate, trivinylbenzene, trivinylcyclohexane, vinyl acetate, vinylbenzyl alcohol, 4-vinyl-1-cyclohexene 1,2-epoxide, vinylformamide, vinylnaphthalene, 2-vinyloxirane, and vinyltoluene. In preferred sorbents, the formed polyol is a diol.

In another embodiment, polymeric sorbents are prepared from the reaction of a cross-linker with vinyl acetate and subsequently modified to form a bead containing polyol groups. The reaction may be a copolymerization, or a one-pot reaction in which vinyl acetate is added once initial polymerization has nearly completed, utilizing unused initiator to begin a second free-radical polymerization to add vinyl acetate groups to the surface of the polymer beads. The subsequent modification of the vinyl acetate containing polymer includes, in order: hydrolysis to convert acetate groups into hydroxyl groups, reaction with epichlorohydrin to form polymer beads containing epoxide groups, and ring-opening to convert epoxide groups into polyol groups. In preferred embodiments, polyols are diols.

Some embodiments of the invention involve direct synthesis of polymeric beads containing epoxide groups, followed by ring-opening of epoxide groups to form polyols. One or more of the following polymerizable vinyl monomer containing epoxide groups can be polymerized in the presence of cross-linker and monomer to yield polymeric beads containing above mentioned functionalities: allyl glycidyl ether, 3,4-dihydroxy-1-butene, 3,4-epoxy-1-butene, 1,2-epoxy-9-decene, 1,2-epoxy-5-hexene, glycidyl methacrylate, 4-vinyl-1-cyclohexene 1,2-epoxide, and 2-vinyloxirane. Vinyl monomers containing epoxide groups can also be copolymerized with hemocompatible monomer (NVP. 2-HEMA, etc.) to yield hemocompatible beads containing epoxide groups. In preferred embodiments; the polyols are diols.

Still other embodiments consist of hypercrosslinked polymeric sorbents containing polyol groups on the beads' surfaces. This can be accomplished via free-radical or S_{N}2 type chemistries. The chemical modification of the surface of sorbent beads, which is the case in the above modification, is facilitated by the remarkable peculiarity of the hypercrosslinked polystyrene; namely, that the reactive functional groups of the polymer are predominantly located on its surface. The hypercrosslinked polystyrene is generally prepared by crosslinking polystyrene chains with large amounts of bifunctional compounds, in particular, those bearing two reactive chloromethyl groups. The latter alkylate, in a two-step reaction, two phenyl groups of neighboring polystyrene chains according to Friedel-Crafts reaction, with evolution of two molecules of HCl and formation of a cross bridge. During the crosslinking reaction, the three-dimensional network formed acquires rigidity. This property gradually reduces the rate of the second step of the crosslinking reaction, since the reduced mobility of the second pendant functional group of the initial crosslinking reagent makes it more and more difficult to add an appropriate second partner for the alkylation reaction. This is especially characteristic of the second functional groups that happen to be exposed to the surface of the bead. Therefore, of the pendant unreacted chloromethyl groups in the final hypercrosslinked polymer, the largest portion, if not the majority of the groups, are located on the surface of the bead (or on the surface of pores). This circumstance makes it possible to predominantly modify the surface of the polymer beads by involving the above chloromethyl groups into various chemical reactions that allow attachment of biocompatible and hemocompatible monomers, and/or cross-linkers or low molecular weight oligomers. The subsequent introduction of hydroxyl groups, followed by reaction with epichlorohydrin, results in the polymer sorbent containing epoxide groups on the beads' surfaces. These epoxide groups can then be ring-opened to form polyol groups. In some preferred embodiments, the polyols are diols.

In other embodiments, hypercrosslinked polystyrene containing pendant unreacted chloromethyl groups is directly modified in the presence of one or more of the following reagents to form sorbent polymer beads containing polyols on the beads' surfaces (or on the surface of pores): (+)-3-amino-1,2-propanediol, glycerol, and other polyols. In preferred embodiments, the polyols are diols.

Still in other embodiments, the surface coating biocompatibility and hemocompatibility agent, poly(vinyl alcohol), also acts as the polyol functional group.

In some other embodiments, sorbents include cross-linked polymeric material derived from the reaction of a cross-linker with one or more of the following polymerizable monomers, then subsequently reacted with a polymerizable zwitterionic monomer in the presence of a free radical initiator: acrylonitrile, allyl glycidyl ether, butyl acrylate, butyl methacrylate, cetyl acrylate, cetyl methacrylate, 3,4-dihydroxy-1-butene, dipentaerythritol diacrylate, dipentaerythritol dimethacrylate, dipentaerythritol tetraacrylate, dipentaerythritol tetramethacrylate, dipentaerythritol triacrylate, dipentaerythritol trimethacrylate, divinylbenzene, divinylformamide, divinylnaphthalene, divinylsulfone, 3,4-epoxy-1-butene, 1,2-epoxy-9-decene, 1,2-epoxy-5-hexene, ethyl acrylate, ethyl methacrylate, ethylstyrene, ethylvinylbezene, glycidyl methacrylate, methyl acrylate, methyl methacrylate, octyl acrylate, octyl methacrylate, pentaerythritol diacrylate, pentaerythritol dimethacrylate, pentaerythritol tetraacrylate, pentaerythritol tetramethacrylate, pentaerythritol triacrylate, pentaerythritol trimethacrylate, styrene, trimethylolpropane diacrylate, trimethylolpropane dimethacrylate, trimethylolpropane triacrylate, trimethylolpropane trimethacrylate, trivinylbenzene, trivinylcyclohexane, vinyl acetate, vinylbenzyl alcohol, 4-vinyl-1-cyclohexene 1,2-epoxide, vinylformamide, vinylnaphthalene, 2-vinyloxirane, and vinyltoluene. Polymerizable zwitterionic monomers include one, or more, of the following: 2-Acrylamido-2-methyl-1-propanesulfonic acid sodium salt, [3-(Acryloylamino)propyl]-trimethylammonium chloride, 3-[[2-(Acryloyloxy)ethyl]-dimethylammonio]-propionate, [2-(Acryloyloxy)ethyl]-dimethyl-(3-sulfopropyl)-ammonium hydroxide, 2-Acryloyloxyethyl phosphorylcholine, [3-(Methacryloylamino)propyl]-trimethylammonium chloride, 3-[[2-(Methacryloyloxy)ethyl]-dimethylammonio]-propionate, [2-(Methacryloyloxy)ethyl]-dimethyl-(3-sulfopropyl)-ammonium hydroxide, and 2-Methacryloyloxyethyl phosphorylcholine.

In one embodiment, the polymers of this invention are made by suspension polymerization in a formulated aqueous phase with free radical initiation in the presence of aqueous phase dispersants that are selected to provide a biocompatible and a hemocompatible exterior surface to the formed polymer beads. In some embodiments, the beads are made porous by the macroreticular synthesis with an appropriately selected porogen (pore forming agent) and an appropriate time-temperature profile for the polymerization in order to develop the proper pore structure.

In another embodiment, polymers made by suspension polymerization can be made biocompatible and hemocompatible by further grafting of biocompatible and hemocompatible monomers or low molecular weight oligomers. It has been shown that the radical polymerization procedure does not consume all the vinyl groups of DVB introduced into copolymerization. On average, about 30% of DVB species fail to serve as crosslinking bridges and remain involved in the network by only one of two vinyl groups. The presence of a relatively high amount of pendant vinyl groups is therefore a characteristic feature of the adsorbents. It can be expected that these pendant vinyl groups are preferably exposed to the surface of the polymer beads and their macropores, if present, should be readily available to chemical modification. The chemical modification of the surface of DVB-copolymers relies on chemical reactions of the surface-exposed pendant vinyl groups and aims at converting these groups into more hydrophilic functional groups. This conversion via free radical grafting of monomers and/or cross-linkers or low molecular weight oligomers provides the initial hydrophobic adsorbing material with the property of hemocompatibility.

In yet another embodiment, the radical polymerization initiator is initially added to the dispersed organic phase, not the aqueous dispersion medium as is typical in suspension polymerization. During polymerization, many growing polymer chains with their chain-end radicals show up at the phase interface and can initiate the polymerization in the dispersion medium. Moreover, the radical initiator, like benzoyl peroxide, generates radicals relatively slowly. This initiator is only partially consumed during the formation of beads even after several hours of polymerization. This initiator easily moves toward the surface of the bead and activates the surface exposed pendant vinyl groups of the divinylbenzene moiety of the bead, thus initiating the graft polymerization of other monomers added after the reaction has proceeded for a period of time. Therefore, free-radical grafting can occur during the transformation of the monomer droplets into polymer beads thereby incorporating monomers and/or cross-linkers or low molecular weight oligomers that impart biocompatibility or hemocompatibility as a surface coating.

As used herein, the term “sorbent” includes adsorbents and absorbents.

As used herein, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The term “biocompatible” is defined to mean the sorbent is capable of coming in contact with physiologic fluids, living tissues, or organisms, without producing unacceptable clinical changes during the time that the sorbent is in contact with the physiologic fluids, living tissues, or organisms.

The term “hemocompatible” is defined as a condition whereby a biocompatible material when placed in contact with whole blood or blood plasma results in clinically acceptable physiologic changes.

As used herein, the term “physiologic fluids” are liquids that originate from the body and can include, but are not limited to, nasopharyngeal, oral, esophageal, gastric, pancreatic, hepatic, pleural, pericardial, peritoneal, intestinal, prostatic, seminal, vaginal secretions, as well as tears, saliva, lung, or bronchial secretions, mucus, bile, blood, lymph, plasma, serum, synovial fluid, cerebrospinal fluid, urine, and interstitial, intracellular, and extracellular fluid, such as fluid that exudes from burns or wounds.

As used herein, the term “sorbent” includes adsorbents and absorbents.

For purposes of this invention, the term “sorb” is defined as “taking up and binding by absorption and/or adsorption”.

The term “dispersant” or “dispersing agent” is defined as a substance that imparts a stabilizing effect upon a finely divided array of immiscible liquid droplets suspended in a fluidizing medium.

The term “heparin mimicking polymer” refers to any polymer that possesses the same anticoagulant and/or antithrombogenic properties as heparin.

The term “macroreticular synthesis” is defined as a polymerization of monomers into polymer in the presence of an inert precipitant which forces the growing polymer molecules out of the monomer liquid at a certain molecular size dictated by the phase equilibria to give solid nanosized microgel particles of spherical or almost spherical symmetry packed together to give a bead with physical pores of an open cell structure [U.S. Pat. No. 4,297,220, Meitzner and Oline, Oct. 27, 1981; R. L. Albright, Reactive Polymers, 4, 155-174(1986)].

The term “hypercrosslinked” describes a polymer in which the single repeating unit has a connectivity of more than two. Hypercrosslinked polymers are prepared by crosslinking swollen, or dissolved, polymer chains with a large number of rigid bridging spacers, rather than copolymerization of monomers. Crosslinking agents may include bis(chloromethyl) derivatives of aromatic hydrocarbons, methylal, monochlorodimethyl ether, and other bifunctional compounds that react with the polymer in the presence of Friedel-Crafts catalysts [Tsyurupa, M. P., Z. K. Blinnikova, N. A. Proskurina, A. V. Pastukhov, L. A. Pavlova, and V. A. Davankov. “Hypercrosslinked Polystyrene: The First Nanoporous Polymeric Material.” Nanotechnologies in Russia 4 (2009): 665-75.]

The following Examples are provided to illustrate some of the concepts described within this disclosure. While the Examples are considered to provide an embodiment, it should not be considered to limit the more general embodiments described herein.

### EXAMPLES

### Example 1—In Vitro Removal of Cytochrome C, an Inflammatory Mediator

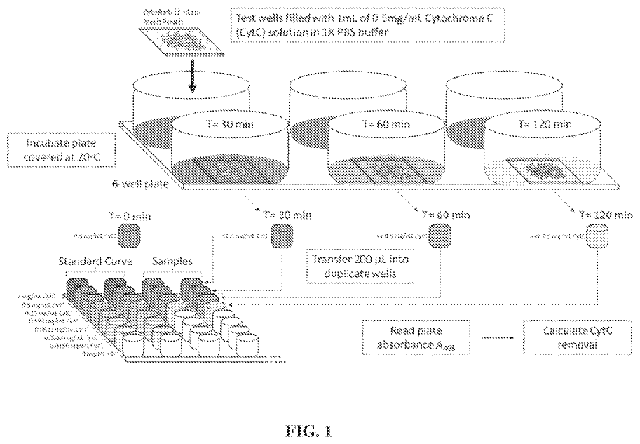

Select wells of a 6 well plate was filled with 1 mL of 0.5 mg/mL Cytochrome C (CytC) solution in 1×PBS buffer, as depicted in FIG. 1.

CytC is a 12 kDa protein that is involved in the electron transport chain for energy production in mitochondria and plays a role in cellular apoptosis. More recently, it has been found in the general blood circulation and interstitial space outside of the cell, released extracellularly due to disease states that can cause mitochondrial and cellular damage, necrosis, and apoptosis. Outside of the cell, cytochrome C acts as a damage-associated molecular pattern (DAMP), or an inflammatory mediator released by damaged tissue, that can then contribute to both systemic and localized inflammation [Eleftheriadis, T., et al, Cytochrome C as a potentially clinical useful marker of mitochondrial and cellular damage”, Front Immunol. 2016; 7:279.]. Removal of cytochrome C and other inflammatory mediators by CytoSorb may help to reduce systemic inflammation and may reduce localized inflammation, such as seen in skin injuries or disease. Cytochrome C is also similar in size to lower molecular weight cytokines and is easy to assay by spectrophotometry due to its colorimetric properties.

A pouch with CytoSorb beads was placed into the wells filled with the 0.5 mg/mL Cytochrome C (CytC) solution in 1×PBS buffer, with one side of the pouch in contact with the CytC solution.

The pouches were allowed to sit for t=30, 60, or 120 minutes without any movement or shaking. After each of these timeframes, it was observed that there was no major loss of liquid in the wells, but there was a loss of color (Cytochrome C) in a time-dependent manner, indicating that the CytC had wicked up and been adsorbed by the CytoSorb beads, which became pink in color.

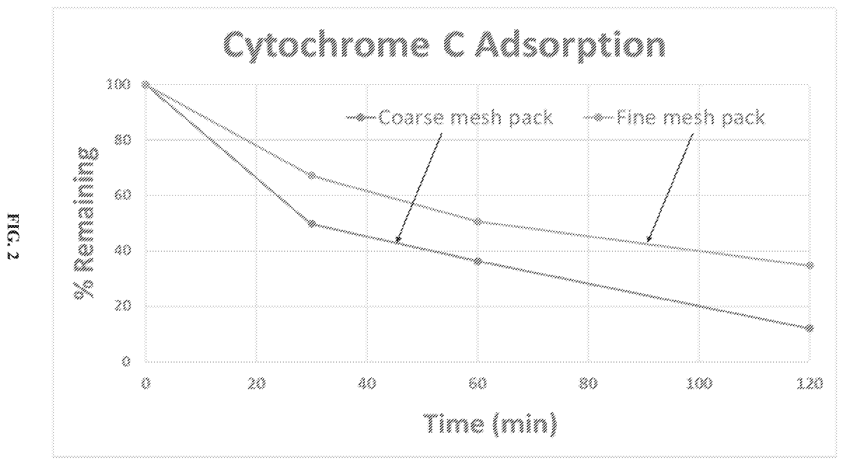

The absorbance of the liquid in the wells was determined using a wavelength of 405 nm (A₄₀₅) and the removal of CytC as a percent of starting concentration was calculated. The results are shown below in Table 1 as well as in FIG. 2.

TABLE 1
In Vitro Removal of CytC

| Time | % CytC Remainingª | % CytC Remainingª |
|---|---|---|
| (minutes) | (Coarse Mesh) | (Fine Mesh) |
| 0 | 100.0% | 100.0% |
| 30 | 49.7% | 67.2% |
| 60 | 36.3% | 50.5% |
| 120 | 12.0% | 34.7% |

ªData in Table 1 is the average of two wells

### Example 2—Animal Testing of Cytochrome C Removal

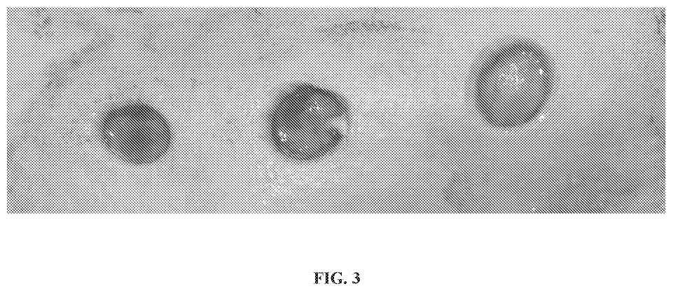

8 mm diameter full thickness skin wounds were made on the backs of dead rats, as shown in FIG. 3. Each wound was filled with about 90 μL of a 3.8 mg/mL CytC solution.

A pouch with CytoSorb beads was pressed against each wound and held using Tegaderm wrap. After about 10 minutes, the pouches were removed from the wound and visually inspected. It was observed that CytC had been adsorbed, as determined by the color transferred to the beads from the solution.

### Example 3—In Vivo Wound Healing

8 mm diameter full thickness skin wounds were created surgically under anesthesia on the backs of rats. The wounds were overlaid with: a) ˜80 μL of CytoSorb beads (test) and covered with Tegaderm; or b) ˜80 μL of non-porous beads made of the same material as CytoSorb (control) and covered with Tegaderm; or c) simply covered with gauze (untreated).

The beads and dressings were changed every two days through day 8 and wounds were observed and photographed on day 0, 2, 4, 6, 8 and 14. After 14 days, animals were sacrificed, and histopathology of the wound site was performed.

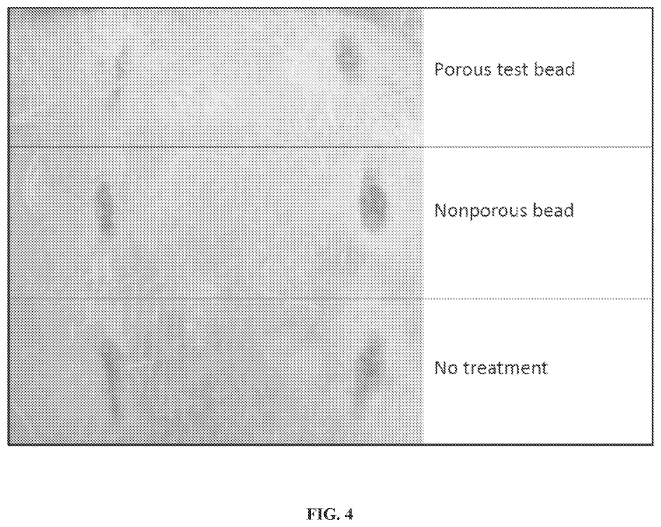

Granulation tissue, a key factor for normal wound healing that aids in wound defect filling, as well as induction of epithelialization, blood vessel growth, extracellular matrix deposition, and cellular recruitment to enable pathogen control, was measured on a scale of 0 (no granulation tissue present) to 4 (marked granulation tissue present). Based on histopathologic examination, the use of porous and non-porous beads in the wound bed led to significantly more granulation tissue (granulation scores of 3.1 and 3.2, respectively) compared to a granulation score of 1.6 for untreated wounds. For porous beads vs untreated, this difference was statistically significant at a p=0.001). For porous beads, the granulation tissue exhibited signs of advancing maturation, with organization of collagen into bundles, mild-moderate overall cellularity, and mild-moderate vascularizaton. The use of beads was associated with a slight delay in wound closure in the rats tested, compared to the untreated control. The healing rate was not substantially different after 14 days as shown in FIG. 4. Moreover, it was observed that epithelial repair and inflammatory responses were similar between wounds treated with test article and untreated wounds. It was also observed that neither the porous beads nor the control beads, both synthesized from the same polymer, induced erythema or edema, supporting that both are biocompatible and do not induce inflammation.

It is possible that the more controlled and slower rate of wound healing and concomitant increase in granulation tissue during treatment could potentially allow for robust differentiation and tissue reconstruction at the wound site, resulting in more thorough Type I collagen fiber deposition and crosslinking, reduced fibrosis, and an increase in abundance of adnexa such that the final dermal architecture more closely resembles uninjured skin, with both enhanced strength and reduced scarring.

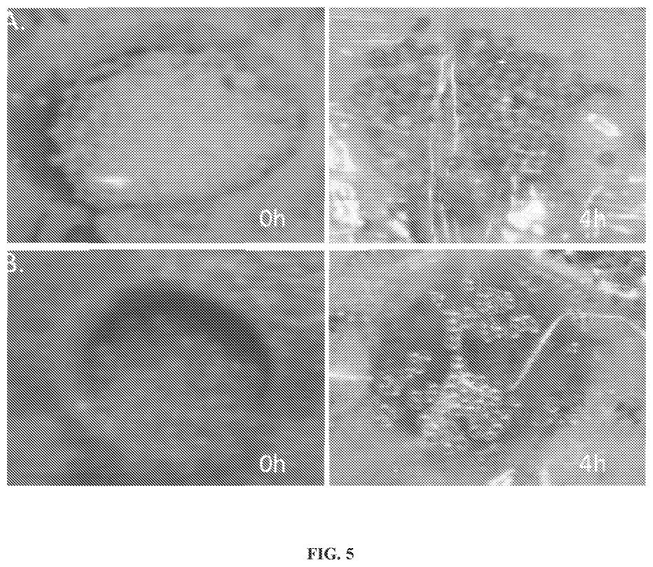

Further, it was observed that the test beads adsorbed/removed various substances from the wound exudate over the course of the experiment, whereas the control, non-porous beads did not. The test beads initially turned dark brown in the initial stages of the wound healing process, whereas the control beads did not, as shown in FIG. 5. Without wishing to be bound by any particular theory, this is presumably due to adsorption of substances such as proteins, like cytokines, inflammatory mediators, hemoglobin, and others. The test beads eventually turned yellow as the wound healing process progressed. Without wishing to be bound by any particular theory, this is presumably due to adsorption of other unknown proteins or pigmented molecules.

Without wishing to be bound by any particular theory, the beads used in Examples 1-3 aided in the selective, topical or transdermal delivery or removal of one or more substances to or from the skin or surface of a human or animal subject.

What is claimed:

1. A method for the selective, topical or transdermal delivery or removal of one or more substances to or from the skin or surface of a human or animal subject, said method comprising topical or transdermal application of a porous, polymeric composition capable of releasing or sorbing said substances, wherein said porous polymer is a particulate material, with particles having an average diameter in the range of approximately 0.1 microns to approximately 0.5 centimeters, and having a plurality of pores with an average pore diameter in a range of approximately 50 Angstroms to approximately 40,000 Angstroms.

2. The method of claim 1, comprising the application of said porous polymeric composition to a wound.

3. The method of claim 2, wherein said wound is a chronic wound, a venous stasis ulcer, an ulcer or ulceration caused by a disease, a traumatic wound, a burn wound, a diabetic wound, or a surgical wound.

4. The method of claim 2, comprising the application of said porous polymeric composition to alter or improve wound healing, reduce scarring, improve tissue remodeling, or reduce inflammation or pain.

5. The method of claim 1, comprising the application of said porous polymeric composition to skin.

6. The method of claim 1, comprising the application of said porous polymeric composition to a skin graft site.

7. The method of claim 1, comprising the dermal application of said porous polymeric composition for plastic, cosmetic, and reconstructive surgery applications.

8. The method of claim 5, where the said skin application is for cosmetic applications.

9. The method of claim 8, where said cosmetic applications includes one or more of skin brightening, cleansing, exfoliating, anti-aging, beautifying, anti-wrinkle, softening, oil reduction, pore cleansing, skin rejuvenation, improving skin discoloration, and reducing fine lines, skin laxity, and skin fragility.

10. The method of claim 5, where the said skin application is for the treatment of one or more dermatologic conditions.

11. The method of claim 5, where the said dermatological conditions include infections, yeast infection, fungal infection, warts, malodorous skin, hyperhidrosis, dandruff, seborrheic dermatitis, skin manifestations of autoimmune diseases, psoriasis, lupus, Lichen planus, dry or oily skin, eczema, atopic dermatitis, contact or allergic dermatitis, tinea, vitiligo, rashes, hives, decubitus ulcers, canker or cold sores, stomatitis, versicolor, pemphigoid, rosacea, skin blotchiness, corns, calluses, ichthyosis vulgaris, keloids, seborrheic keratosis, actinic keratosis, and skin cancer.

12. The method of claim 10, where the said dermatological condition is acne vulgaris.

13. The method of claim 5, where the said skin application alters the activity of immune cells in the skin.

14. The method of claim 12, where a change in activity of immune cells leads to a change in inflammation or aging of the skin.

15. The method of claim 1, where the porous polymeric composition is in the form of a powder, a poultice, a mask, a liquid, a gel paste, a low volume paste, a gel, a dispersion, a slurry, or a suspension.

16. The method of claim 14, wherein said porous polymeric composition is used in conjunction with a cleansing cloth, pad, towelette, or rotary cleansing apparatus.

17. The method of claim 1, wherein said porous polymeric composition also comprises a permeable material or liquid.

18. The method of claim 17, wherein said permeable material is a gauze, mesh, pad, or permeable or semi-permeable membrane in which said porous polymeric material is embedded or enclosed.

19. The method of claim 17, wherein said permeable material is a liquid, gel, lotion, or paste.

20. The method of claim 17, wherein said permeable material contains at least one or more chemicals selected from drugs, medications, vitamins, nitric oxide, nitric oxide donors), minerals, or nutrients.

21. The method of claim 20 where the said chemical is an antibiotic, anti-viral, antifungal, or anti-parasitic medicine.

22. The method of claim 17, wherein said permeable material contains at least one cosmetic ingredient.

23. The method of claim 22, wherein said cosmetic ingredient comprises one or more of the following: glycerin, hyaluronic acid, a hyaluronic acid salt, shea butter, vitamins, vitamin E, vitamin A, vitamin D, vitamin C, and vitamin K.

24. The method of claim 22, wherein said cosmetic ingredient comprises one or more of cleansers, moisturizers, sunscreens, antibiotics, benzoyl peroxide, keratolytic agents, alpha-hydroxy acids, beta-hydroxy acids, retinoic acid, hydroxyquinone, potassium hydroxide, tea extracts, and plant and flower extracts.

25. The method of claim 1, wherein the one or more substances comprise a protein, peptide, glycosylated protein or lipid (e.g. advanced glycation end product), or protein containing molecule.

26. The method of claim 25, wherein the one or more substances comprise a protein-based inflammatory mediator such as a cytokine, toxin, or activated complement.

27. The method of claim 26, wherein the cytokine comprises one or more of TNF-α, IL-1, IL-4, IL-5, IL-6, IL-8, IL-10, IL-12, RANTES, MCP-1, or IP-10.

28. The method of claim 25, wherein said substance is a growth factor.

29. The method of claim 25, wherein said substance is an enzyme.

30. The method of claim 29, wherein said enzyme is a metalloproteinase, a collagenase, an elastase, or a cross-linking enzyme.

31. The method of claim 1, wherein said substance is a non-protein inflammatory mediator such as a prostaglandin, leukotriene, bioactive lipid or histamine.

32. The method of claim 1, wherein said substance is a hormone.

33. The method of claim 1, wherein said substance is a pain mediator.

34. The method of claim 1, wherein said substance is a wax, squalene, fatty acid, triglyceride, or oil, such as sebum, or urushiol oil.

35. The method of claim 1, wherein said porous polymeric composition comprises a mixture of particles having at least two different average diameters.

36. The method of claim 1, wherein said porous polymeric material is biocompatible or does not induce inflammation.

37. The method of claim 1, wherein said porous polymeric material comprises a plurality of pores ranging from 50 Å to 40,000 Å with a pore volume of 0.5 cc/g to 5.0 cc/g, said sorbent having a size of 0.05 mm to 2 cm;
wherein the sorbent has a pore structure such that the total pore volume of pore size in the range of 50 Å to 40,000 Å is greater than 0.5 cc/g to 5.0 cc/g dry sorbent; wherein:
(i) the ratio of the total pore volume of pore diameter in the range of 50 Å to 40,000 Å to the total pore volume of pore diameter in the range of 100 Å to 1,000 Å of the sorbent is smaller than 3:1; or
(ii) the ratio of the total pore volume of pore diameter in the range of 50 Å to 40,000 Å to the total pore volume of pore diameter in the range of 1,000 Å to 10,000 Å of the sorbent is smaller than 2:1; or
(iii) the ratio of the total pore volume of pore diameter in the range of 50 Å to 40,000 Å to the total pore volume of pore diameter in the range of 10,000 Å to 40,000 Å of the sorbent is smaller than 3:1.
